(19) United States
(12) Patent Application Publication
(10) Pub. No.: US 2013/0237973 A1
(43) Pub. Date: Sep. 12, 2013

(54) LASER EMISSION SYSTEM AND ROBOT LASER EMISSION DEVICE COMPRISING THE SAME

(71) Applicants: SNU R&DB FOUNDATION, Seoul (KR); SEOUL OCEAN AQUARIUM CO., LTD., Seoul (KR)

(72) Inventors: Sung Wan KIM, Seoul (KR); Hee Chan KIM, Seoul (KR); Jin Ho CHUNG, Seoul (KR); You Dan KIM, Seoul (KR); Chi Yul YOON, Gyeonggi-do (KR); Seung Woo Noh, Busan (KR); Hyoung Woo Lim, Seoul (KR); Chong Hee Lee, Seoul (KR); Chi Won Lee, Gyeonggi-do (KR); Woo Seok Koh, Seoul (KR)

(21) Appl. No.: 13/778,626

(22) Filed: Feb. 27, 2013

(30) Foreign Application Priority Data
Mar. 9, 2012 (KR) 10-2012-0024650

(51) Int. Cl. A61B 18/20; A61B 19/00

(52) U.S. Cl. CPC A61B 18/203; A61B 19/2203
USPC 606/9

(57) ABSTRACT

The present invention discloses a laser emission system comprising a laser emission device for irradiation a laser to an area of a targeted object; a monitoring device; a display unit for displaying an image captured by the monitoring device; and a control unit connected to the monitoring device and the display unit. Here, the control unit receives the information on a movement direction and a moving distance of the laser emission device and distinguishes between an area emitted with a laser by the laser emission device and an area which is not emitted with a laser, and then displays them on the display unit. Regardless of the perceived skill of the operator, the present invention prevents the irradiation of the area from being omitted and prevents the area from being excessively or insufficiently emitted in the laser treatment.

### BACKGROUND OF THE INVENTION

The present invention relates to a laser emission system, and more particularly, to a laser emission system including a monitoring device and a display unit to prevent an irradiation of the target area from being omitted and prevent the area from being excessively or insufficiently emitted when the laser is emitted to an area of a targeted object.

In most of the commercial laser hair removal (LHR) devices, an area emitted with a laser is about 1.0 cm². As compared with the total area on which a treatment for removing body hair must be performed, the above area is a comparatively small area, and so an operator must move the LHR device to sequentially emit every area by 1.0 cm².

During the treatment for removing body hair utilizing the available LHR device, however, there is no method for verifying whether a treatment has been performed. Thus, the laser treatment for removing body hair utilizing the LHR device completely depends on a perceived skill of a medical operator.

During the treatment for removing body hair, accordingly, there is frequently a region to which the laser is not emitted and a region to which the laser is excessively or insufficiently emitted,

Body hair on a region in which laser emission is omitted or is insufficient is not removed, thereby lowering patient satisfaction of the laser treatment for removing body hair. The laser must then be emitted again to the region on which the laser emission is omitted or is insufficient. Also, if the region is excessively emitted, a patient may be injured.

The above drawbacks are not limited to the LHR device, but to all kinds of therapeutic apparatuses which employ the laser emission device and therefore have in cannon the above problems.

### SUMMARY OF THE INVENTION

The present invention is conceived to solve the above problems, and an object of the present invention is to provide a laser emission system comprising a monitoring device and a display Unit for enabling a quantity of the region corresponding to a preset quantity to be accurately emitted regardless of a perceived skill of an operator and for preventing the irradiation of the region from being omitted and the region from being excessively or insufficiently emitted.

In addition, an object of the present invention is to provide a laser emission system comprising an alarm section for preventing an omission of the laser emission caused by an operator's carelessness.

To perform automatically the laser emission treatment, another object of the present invention is to provide a robot laser emission device comprising the laser emission system according the present inventions which can accurately emit a quantity of the laser, which is preset in advance, to an area which is preset in advance.

To achieve the above objects, the present invention provides a laser emission system comprising a laser emission device for emitting a laser to an area of a targeted object; a monitoring device; a display unit for displaying an image captured by the monitoring device; and a control unit connected to the monitoring device and the display unit; wherein the control unit receives the information having a movement direction and a moving distance of the laser emission device and distinguishes between an area emitted with a laser by the laser emission device and an area which is not emitted with a laser, and then displays both of them on the display unit.

Preferably, the control unit recognizes a movement of the laser emission device by a pattern matching method to distinguish the irradiated area from the unirradiated area.

In addition, the laser emission system of the present invention further comprises a three-axis marker attached to the laser emission device. Here, the monitoring device captures an image of the three-axis marker, and the control unit recognizes a movement of the three-axis marker captured by the monitoring device and receives the information on a movement direction and a moving distance of the laser emission device to distinguish between an area emitted with the laser emission device and an area on which the laser has not been not emitted.

Preferably, the control unit recognizes a movement of the laser emission device in a markless method to distinguish between an area emitted with the laser emission device and an area on which the laser has not been emitted.

Also, the laser emission system of the present invention may further comprise two roller and encoders with the laser emission device. Each encoder measuring rotations of each roller to determine a movement direction and calculate a moving distance of the laser emission device. Preferably, the rollers can be rotated in one axial direction, and the rollers are arranged such that one of the rollers is rotated according a horizontal movement of the laser emission device and the other one of the rollers is rotated according a vertical movement of the laser emission device.

Furthermore, the laser emission system of the present invention may further comprise a ball sensor in the laser emission device and a ball rotation counter which can determine the movement direction and calculate the moving distance of the laser emission device.

Preferably, the laser emission system of present invention may further comprise an accelerometer with the laser emission device. The control unit measures acceleration and calculate the moving distance of the laser emission device by the acceleration signals.

In addition, the laser emission system of the present invention may further comprise an optical moving distance measuring device with the laser emission device. Optical type moving distance measuring device can determine the movement direction and calculate the moving distance of the laser emission device.

Also, the laser emission system according to the present invention may further comprise a laser type moving distance measuring device with the laser emission device. The moving distance measuring device can determine the movement direction and calculate the moving distance of the laser emission device.

Preferably, the laser emission system of the present invention may further comprise a laser emission intensity sensor unit for measuring the emission intensity of the laser which is emitted to each point of the area of the targeted object. The control unit displays the emission intensity of the laser in each point, which is measured by the laser emission intensity sensor unit on the display unit.

Here, the laser emission intensity sensor unit can be a timer device which can measure the duration of laser emission at each point.

Also, the laser emission intensity sensor unit may be a thermometer which can measure a temperature of each point.

In addition, the laser emission intensity sensor unit may be a color-measuring device which can measure a skin color of each point to detect the change of skin color caused be laser absorption.

The laser emission system of the present invention may further comprise an alarm unit. The control unit controls the alarm unit to generate a vibration or a sound if the intensity of the laser emission exceeds the preset intensity or if some of the area was omitted during the treatment.

In particular, the laser emission device can be a hand-held type laser emission device.

The laser emission system as Mentioned above can he combined with the robot arm. The control unit controls the robot arm to move the laser emission device to region of interest area.

The present invention prevents the untreated and being excessively and insufficiently treated region in the laser treatment regardless of the skill of the operator.

In addition, the operator can complete a laser treatment through the robot laser emission device by setting the robot to move and emit the laser in targeted area.

Other objects, features and advantages of the present invention will become more apparent from the following detailed description when read in conjunction with the accompanying drawings.

### BRIEF DESCRIPTION OF THE DRAWINGS

FIG. 1 is a perspective view of illustration according to the present invention which an operator use a laser emission system to a targeted object;

FIG. 2 is a perspective view of a hand-held type laser hair removal device which is one example of the laser emission device; and

FIG. 3 is a perspective view of a robot based laser emission device including the laser emission system according to the present invention.

### DESCRIPTION OF SPECIFIC EMBODIMENTS

The terminology “pattern matching” mentioned later in the detailed description indicates the algorithm for detecting a position of a laser emission device in the captured image by the monitoring device. The algorithm using an image of the laser emission device as a pattern for comparing with captured image by a monitoring device. Monitoring device can detect a movement of the laser emission device by using pattern matching algorithm.

The terminology “markless method” mentioned later in the detailed description indicates the method for detecting a position of a laser emission device. The “markless method” compares a three-dimensional shape of the laser emission device with the captured image by monitoring device. With this method, control unit can recognize the position of the laser emission device in the space without a specific optical marker.

Here in after, a laser emission system of the presented invention is explained in detail with the drawings.

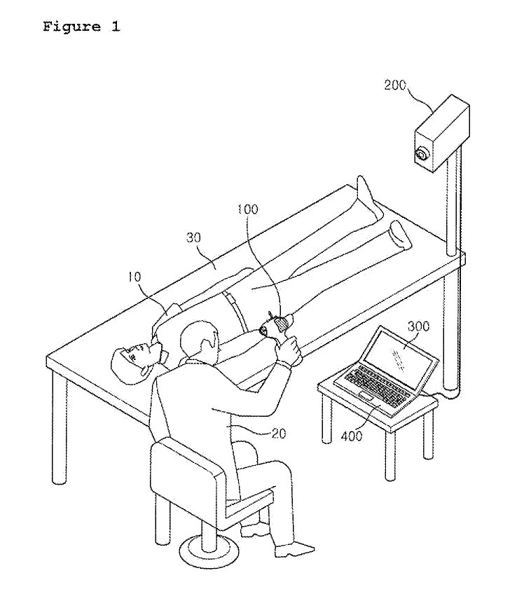

FIG. 1 is a perspective view illustration that an operator 20 emits a laser to a targeted object 10 lying on a bed 30 through a laser emission system. As shown in FIG. 1, the laser emission system of the present invention may include a laser emission device 100, a monitoring device 200, a display unit 300 and a control unit 400.

The laser emission device 100 indicates all kinds of devices which can emit the laser to the targeted object.

Preferably, the laser emission device 100 is a hand-held type laser hair removal device 1000.

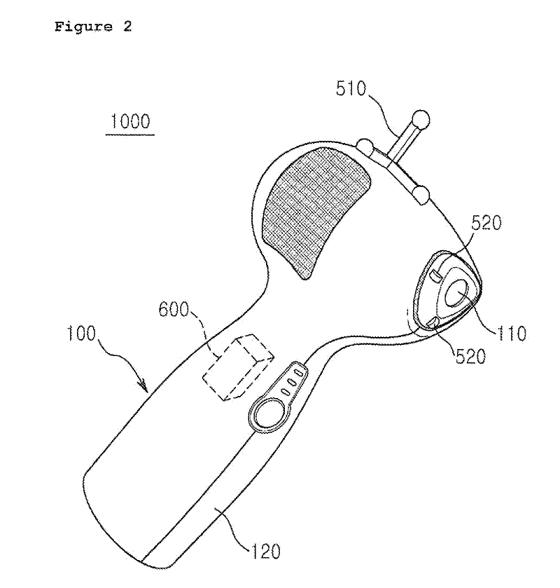

As shown in FIG. 2, the hand-held type laser hair removal device 1000 may comprise a laser emission unit 110 and a gripper 120.

The laser is emitted on the targeted object 10 through the laser emission unit 110, and the gripper 120 is a part on which a hand of the operator 20 is placed.

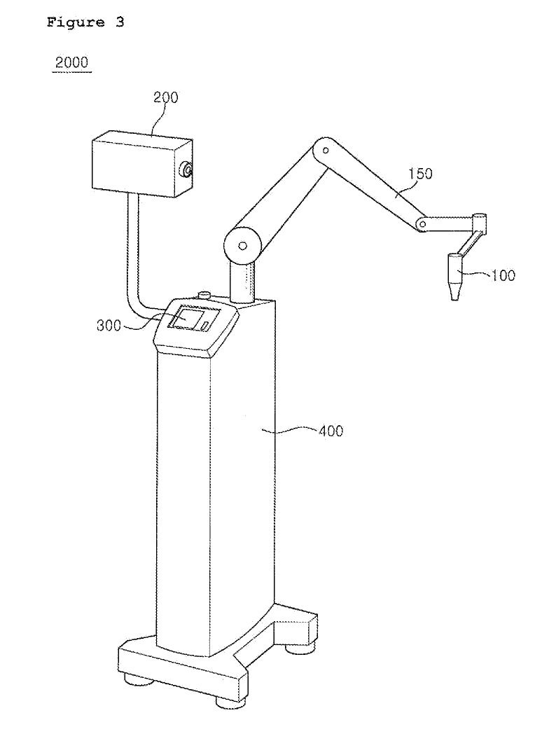

For example, a camera may be used as the monitoring device 200. As the monitoring device, however, it is possible to employ any device which can obtain an image. As shown in FIG. 1 and FIG. 3, the monitoring device 200 should be installed to obtain a scene of the targeting area and treatment circumference.

For example, a monitor of a computer may be employed as the display unit 300. However, any device which can display a captured image by the monitoring device 200 and a processed image by the control unit 400 can be utilized as the display unit. As shown in FIG. 1 and FIG. 3, it is preferable to put the display unit 300 to a good position for the purpose of the operator 20 can observe the display unit during the treatment. In addition, it is possible to mount a small-sized display unit to the hand-held type laser hair removal device 1000 to enable the operator to obtain the information of treatment status during the treatment.

For example, hardware and software of the computer may be used as the control unit 400. The control unit 400 is electrically connected to the monitoring device 200 and the display unit 300. In this case, FIG. 1 shows that the control unit 400 is connected to the monitoring device 200 and the display unit 300 via wires. However, the control unit can be connected to the monitoring device and the display unit in a wireless communication protocols.

The control unit 400 displays a captured image by the monitoring device 200 on the display unit 300. An area which is already treated and not treated of the laser by the laser emission device 100 are distinguished and then displayed on the display unit. In addition, it is possible to quantitatively display the intensity of emitted laser on each place. Thus, the operator 20 can distinguish between the area which is already operated and the area to be operated only by observing the display unit 300, and the operator can also find easily an area which has not been emitted with the laser and an area which is excessively or insufficiently emitted with the laser.

Preferably, the control unit 400 employs a pattern matching algorithm for distinguishing between the irradiated area and the unirradiated area by the laser emission device 100. The control unit 400 detects a movement of the laser emission device 100 using the pattern matching algorithm and stores this movement to distinguish between the irradiated and unirradiated area in this case, a two-dimensional image is provided on the display unit 300.

In addition, the control unit 400 preferably employs a markless method for distinguishing between the irradiated and unirradiated area by the laser emission device 100. In this case, the control unit 400 can detect a movement of the laser emission device 100 using the markless method and store this movement therein to distinguish between the irradiated and unirradiated area and to display them on the display unit 300 in the form of a three-dimensional image.

### The First Embodiment

The laser emission system according to the present invention comprises the laser emission device 100, the monitoring device 200, the display unit 300, the control unit 400, and a three-axis marker 510.

A structure and function of each of the laser emission device 100, the monitoring device 200, the display unit 300 and the control unit 400 in this embodiment are the same as those described in the previous description, and so the detailed description thereof is skipped.

As shown in FIG. 2, the three-axis marker 510 is formed by fixedly connecting ends of three linear-shaped bars to each other, and the adjacent two bars are perpendicular to each other. Although, FIG. 2 illustrates the three-axis marker 510 attached to the hand-held type laser hair removal device 1000, the three-axis marker can be mounted to any kind of the laser emission device 100.

The monitoring device 200 captures the three axis marker 510 and a target area. The control unit 400 recognizes a movement of the three-axis marker 510 captured by the monitoring device 200 to distinguish between an irradiated area and unirradiated area by the laser emission device 100. Through the above process, the control unit 400 can distinguish between the irradiated area and unirradiated area by the laser emission device 100 and then displays them on the display unit 300.

### The Second Embodiment

The laser emission system of the present invention comprises the laser emission device 100, the monitoring device 200, the display unit 300, the control unit 400, a roller 520 and an encoder (not shown).

A structure and function of each of the laser emission device 100, the monitoring device 200, the display unit 300 and the control unit 400 in this embodiment are the same as those described in the previous description, so the detailed description thereof is skipped.

The roller 520 can be rotated in only one axial direction.

Accordingly, by arranging two rollers on the laser emission device 100 in the vertical and horizontal directions, respectively, all of the horizontal movements and vertical movements of the laser emission device 100 can be detected.

FIG. 2 is illustration of two rollers 520, one of them being arranged in the horizontal direction and the other one being arranged in the vertical direction. For example, the rollers are arranged around the laser emission unit 110 so as to allow the rollers 520 to be in contact with the targeted object 10 when the targeted object 10 is treated by the laser.

In a case where the operator 20 moves the laser emission device 100, two rollers 520 are rotated according to the vertical and horizontal movement of the laser emission device, respectively, and the encoder receives all the rotation degrees of two rollers 520 to determine two-dimensional movement direction and calculate moving distance.

The information of movement direction and moving distance by the above process are transmitted to the control unit 400, and the control unit stores the transmitted information of movement direction and moving distance. The control unit then distinguishes between the irradiated area and unirradiated area by the laser emission device 100, and then displays them on the display unit 300.

The above process can be performed by the encoder included control unit 400.

### The Third Embodiment

The laser emission system of the present invention comprises the laser emission device 100, the monitoring device 200, the display unit 300, the control unit 400, a ball (not shown) and a counter (not shown).

A structure and function of each of the laser emission device 100, the monitoring device 200, the display unit 300 and the control unit 400 in this embodiment are the same as those described in the above prior descriptions, and so the detailed description thereof is skipped.

Similar to the rollers 520 in the second embodiment, for example, the balls are arranged around the laser emission unit 110 so as to allow the balls to be in contact with the targeted object 10 when the targeted object 10 is treated with the laser.

Unlike the rollers 520 in the second embodiment, the rotation of the ball is not restricted in one axial direction. Thus, although only one ball is provided on the laser emission device 100, both the horizontal movement and the vertical movement can be detected.

For example, the counter may include two rollers moved according to a movement of the ball to receive a horizontal movement and a vertical movement divided from the movement of the ball.

In a case where the operator 20 moves the laser emission device 100, the ball is rotated in a state where the ball is in contact with the targeted object 10. The rotation of the ball is divided into a vertical rotation and a horizontal rotation by the two rollers of the counter. The counter determines the movement direction and calculates moving distance by using the rotation of the ball.

The control unit 400 receives and stores the movement direction and moving distance of the laser emission device 100 transmitted from the counter, and the control unit distinguishes between the irradiated area and unirradiated area by the laser emission device 100, and then displays them on the display unit 300.

The above process can be performed by the counter process included control unit 400.

### The Fourth Embodiment

The laser emission system of the present invention comprises the laser emission device 100, the monitoring device 200, the display unit 300, the control unit 400 and an accelerometer (not shown).

A structure and function of each of the laser emission device 100, the monitoring device 200, the display unit 300 and the control unit 400 in this embodiment are the same as those described in the previous description, and so the detailed description thereof is skipped.

The accelerometer is mounted to the laser emission device 100 to measure an acceleration of the laser emission device 100 when the laser emission device is moved. The measured acceleration is transmitted to the control unit 400, and the control unit 400 determines movement direction and calculates a moving distance of the laser emission device by integrating twice. The control unit 400 utilizes the calculated moving distance of the laser emission device to distinguish between the irradiated area and unirradiated area by the laser emission device 100 and then displays them on the display unit 300.

### The Fifth Embodiment

The laser emission system of the present invention comprises the laser emission device 100, the monitoring device 200, the display unit 300, the control unit 400 and an optical type moving distance measuring device (not shown).

A structure and function of each of the laser emission device 100, the monitoring device 200, the display unit 300 and the control unit 400 in this embodiment are the same as those described in the previous description, and so the detailed description thereof is skipped.

For example, the optical type moving distance measuring device includes a LED (light emitting diode), an optical sensor and a digital signal processing (DSP) chip. The light emission from the LED is reflected by the targeted object 10 and transmitted to the optical sensor. Then, the digital signal processing chip analyzes the transmitted light to the optical sensor to calculate a moving distance.

The calculated moving distance is transmitted to and stored in the control unit 400, and the control unit 400 distinguishes between the irradiated area and unirradiated area by the laser emission device 100, and then displays them on the display unit 300.

Preferably, the optical type moving distance measuring device may be substituted with a laser type moving distance measuring device not shown). The laser type moving distance measuring device is distinguished from the optical type moving distance measuring device in that the laser type moving distance measuring device emits the laser to the targeted object 10 and receives the reflection of the laser.

A utilization of the laser type moving distance measuring device is advantageous in that sensitivity is higher than that obtained by the optical type moving distance measuring device. It is possible to accurately measure the moving distance even if the targeted object 10 has a curved shape.

The laser emission system according to the first to fifth embodiments may further include a laser emission intensity sensor unit 600.

For example, the laser emission intensity sensor unit 600 may be arranged within the laser emission device 100 as shown in FIG. 2.

The laser emission intensity sensor unit 600 can measure the laser intensity which is emitted to each point of the area of the targeted object 10.

Preferably, the laser emission intensity sensor unit 600 may be a timer device (not shown).

The timer device can measure a duration time by which the laser emission device 100 emits the laser to each point to measure the emission intensity the laser. If the intensity of the laser emission device is constantly preset in advance, the total intensity of the emitted laser can be easily calculated by multiplying the measured time by the preset intensity.

Preferably, the laser emission intensity sensor unit 600 may be a thermometer (not shown).

The thermometer can measure a temperature at each point at which the laser emission device 100 emits the laser, to measure the laser emission intensity of the laser.

Preferably, the laser emission intensity sensor unit 600 may be a color-measuring device (not shown).

The color-measuring device can detect a color of each point at which the laser emission device 100 emits the laser, to measure the laser emission intensity of the laser.

On the display unit 300, the control unit 400 displays the laser emission intensity for each point measured by the laser emission intensity sensor unit 600.

Through the display unit 300, accordingly, the operator 20 can prevent the omitted and excessively or insufficiently emitted region.

In addition, the laser emission system according to the first to fifth embodiments may include an alarm unit (not shown in drawings).

Any device which can generate a vibration or a sound to allow the operator 20 to notify can be employed as the alarm unit.

As soon as the intensity of the laser exceeds the preset intensity, the control 400 can notify the operator 20 with a vibration or a sound through the alarm unit. In addition, some of the area preset in advance not to be treated by the laser has been treated, the power supplied to the laser emission device 100 is shut off. Similarly, the moving distance of the laser emission device exceeds the preset distance; it is possible to notify the operator 20 with a vibration or a sound.

Preferably, it is possible to notify the operator 20 by displaying a message window on the display unit 300.

Robot Laser Emission Device

Here in after, laser emission device can be combined with the robot arm according to the present invention is illustrated in detail with referred to FIG. 1.

The robot combined laser emission device according to the present invention may include the laser emission device 100, the monitoring device 200, the display unit 300 and the control unit 400.

The robot combined laser emission device may include the structures according to the first to fifth embodiments.

Furthermore, the robot combined laser emission device may include the laser emission intensity sensor unit 600.

The robot combined laser emission device may include a robot arm 150.

The operator 20 can set a target area to be treated with the laser and an intensity of the laser to be treated to each point in the control unit 400 in advance. The control unit 400 can control the robot arm 150 according to the target area and the intensity of a laser preset in advance to automatically emit the laser to the targeted object 10.

Although the preferred embodiments of the present invention have been disclosed for illustrative purposes, the present invention is not limited thereto, and those skilled in the art will appreciate that various modifications, additions and substitutions are possible, without departing from the scope and spirit of the invention as disclosed in the accompanying claims. Also, the equivalents of these modifications and changes will be within the scope of the present, invention.

### REFERENCE NUMERALS

10: targeted object

20: operator

30: bed

100: laser emission device

110: laser emission unit

120: gripper

150: robot arm

200: monitoring device

300: display unit

400: control unit

510: three-axis marker

520: roller

600: laser emission intensity sensor unit

1000: hand-held type laser hair removal device

2000: robot combined laser emission device

What is claimed is:

1. A laser emission system, comprising
a laser emission device for emitting a laser to an area of a targeted object;
a monitoring device;
a display unit for displaying an image captured by the monitoring device; and
a control unit connected to the monitoring device and the display unit;
wherein the control unit receives the information on a moving direction and distance travelled by the laser emission device and distinguishes the irradiated area from the unirradiated area, and then displays them on the display unit.

2. The laser emission system of claim 1, wherein the control unit recognizes a movement of the laser emission device by using a pattern matching algorithm to distinguish the irradiated from the unirradiated area.

3. The laser emission system of claim 1, further comprising a three-axis marker in the laser emission device, wherein the monitoring device captures an image of the three-axis marker, while the control unit recognizes the movement of the three-axis marker captured by the monitoring device and receives the information on the moving direction and distance travelled by the laser emission device to distinguish the irradiated from the unirradiated area

4. The laser emission system of claim 1, wherein the control unit recognizes the movement of the laser emission device by a markless method to distinguish the irradiated from the unirradiated area.

5. The laser emission system of claim 1, further comprising two rollers that can rotate around only a single axis in the laser emission device and an encoder that determines the moving direction and distance travelled by the laser emission device based on the rotating movement of the two rollers, wherein one of the rollers is positioned to rotate by the horizontal movement of the laser emission device and the other roller is positioned to rotate by the vertical movement of the laser emission device.

6. The laser emission system of claim 1, further comprising a ball sensor in the laser emission device and a ball rotation counter that determines the moving direction and distance travelled by the laser emission device based on the rotating movement of the ball.

7. The laser emission system of claim 1, further comprising an accelerometer in the laser emission device, wherein the control unit recognizes an acceleration signal measured by the accelerometer and calculates the distance travelled by the laser emission device by performing double integration of the acceleration signal.

8. The laser emission system of claim 1, further comprising an optical distance measuring device in the laser emission device for determining the moving direction and distance travelled by the laser emission device.

9. The laser emission system of claim 1, further comprising a laser type-distance measuring device moving distance measuring device in the laser emission device and for determining the moving direction and distance travelled by the laser emission device.

10. The laser emission system of claim 1 further comprising a laser emission intensity sensor unit for measuring the emission intensity of the laser which is emitted to each point on the area of the targeted object, wherein the control unit displays the emission intensity of the laser measured for each point by the laser emission intensity sensor unit on the display unit.

11. The laser emission system of claim 10, wherein the laser emission intensity sensor unit has a function of timer that can measure the duration of laser emission at each targeted point.

12. The laser emission system of claim 10, wherein the laser emission intensity sensor unit has a function of thermometer that can measure the temperature of each targeted point.

13. The laser emission system of claim 10, wherein the laser emission intensity sensor unit has a function of color-measuring device that can detect a skin color of each targeted point.

14. The laser emission system of claim 10, further comprising an alarm unit, wherein the control unit controls the alarm unit for making vibration or sound when the intensity of the laser emission exceeds the preset level of intensity or when some of the target area is omitted during the laser emission process.

15. The laser emission system of claim 10, wherein the laser emission device is a handheld type laser emission device.

16. A robot laser emission device comprising:
a laser emission device for emitting a laser to an area of a targeted object;
a monitoring device;
a display unit for displaying an image captured by the monitoring device;
a control unit connected to the monitoring device and the display unit;
wherein the control unit receives the information on a moving direction and distance travelled by the laser emission device and distinguishes the irradiated area from the unirradiated area, and then displays them on the display unit;
a laser emission intensity sensor unit for measuring the emission intensity of the laser which is emitted to each point on the area of the targeted object, wherein the control unit displays the emission intensity of the laser measured for each point by the laser emission intensity sensor unit on the display unit; and
a robot arm, wherein the control unit controls the robot arm to move the laser emission device to the region of target area.
